(19) United States
(12) Patent Application Publication
(10) Pub. No.: US 2020/0129094 A1
(43) Pub. Date: Apr. 30, 2020

(54) DIAGNOSTIC HEARING HEALTH ASSESSMENT SYSTEM AND METHOD

(71) Applicant: Steven Brian Levine, Weston, CT (US)

(72) Inventor: Steven Brian Levine, Weston, CT (US)

(21) Appl. No.: 16/493,817

(22) PCT Filed: Mar. 6, 2018
(86) PCT No.: PCT/US2018/021117

§ 371 Date: Sep. 13, 2019

Related U.S. Application Data
(60) Provisional application No. 62/471,766, filed on Mar. 15, 2017.

(51) Int. Cl. A61B 5/12; A61B 1/227; A61B 1/00; A61B 5/00; G16H 50/20; G16H 40/67; G16H 15/00; G16H 50/50; G16H 30/20

(52) U.S. Cl. CPC A61B 5/123; A61B 1/227; A61B 1/00011; A61B 1/00009; A61B 5/126; G16H 30/20; G16H 50/20; G16H 40/67; G16H 15/00; G16H 50/50; A61B 5/7465

(57) ABSTRACT

A system and a method for a healthcare provider to easily, accurately and efficiently provide a diagnostic hearing assessment, for their patients, in a way that accurately detects hearing loss and provides recommendations for seeking further assistance from remotely-located ear specialists is provided.

### CROSS-REFERENCE TO RELATED APPLICATIONS

This application claims the benefit of U.S. Provisional Application No. 62/471,766 filed Mar. 15, 2017, which is incorporated herein by reference in its entirety.

### FIELD

A system and method for diagnosing hearing loss and recommending treatment is generally described. In particular, a system that enables a healthcare provider to administer diagnostic hearing assessments, and consult with a remotely-located ear specialist having expertise in disorders of the ear and/or hearing is described.

### BACKGROUND

Incidences of hearing loss have been steadily increasing in the United States. In 1989, the reported incidence of hearing loss in the United States was 266 per thousand households, 283 per thousand households in 2004, and 295 per thousand households in 2008. In 2008, 43.25 million people reported hearing difficulty. Between 2004 and 2008, the hearing impaired population grew 8.8% compared to a 4.5% increase in US households. Furthermore, the percent of the population that acknowledged a hearing loss grew from 10% in 1989 to 11.3% in 2008.

People with hearing loss rarely acknowledge the disorder, which may lead to a large number of people remaining undiagnosed. From the time hearing loss is first noticed, the average hearing impaired person delays seven years before seeking assistance. Some reasons for the delay may include the perception by the hearing impaired that hearing aid devices geared toward treating hearing loss are expensive, thus they choose to not inquire about a hearing analysis. Others may fear ridicule and taunting by family and friends if they use hearing aid devices, and some others choose to avoid hearing assessments because acknowledging hearing loss may mean an acknowledgment that they are aging, and their bodies are unable to do things they once took for granted.

Technology used in hearing aids is improving rapidly in a manner parallel to other chip-based consumer goods. Newest technologies remain relatively expensive, but slightly older technologies are more affordable. Furthermore, continuous improvement has been made in designs to make hearing aids less cumbersome and noticeable in the ear, along with better ability to eliminate background and/or undesirable noises. Therefore, with early identification and the availability of improved treatment options, an increased number of hearing impaired individuals can benefit from hearing assistance.

Conventional identification systems and methods often include a physician or other healthcare providers who perform hearing tests, utilizing various devices to provide some guidance on the matter. Furthermore, if under such conditions a hearing disorder is identified, the patients are often not referred in an expeditious and efficient manner for diagnosis and/or treatment. In some instances, the healthcare provider may be unable to provide such guidance, and have to refer the patient to an ear specialist for further guidance and/or a more accurate/informative diagnosis, and treatment plan. Once the patient receives the referral, the patient may have to schedule an appointment with the ear specialist to go into yet another medical office for further assessment or in-person hearing health examination. While such medical offices may be in close proximity to either the healthcare provider's office and/or the patient's residence, the ear specialist's medical may require the patient to either wait a long time to get an appointment and/or require the patient to travel long distances. This may be particularly cumbersome for patients experience substantial hearing loss and/or patients who have difficulty travelling long distances, such as the elderly.

According to the disadvantages described above, there is a need for a system and a method that enables a wide range of adult healthcare providers to administer a diagnostic hearing assessment and consult with a remotely-located ear specialist. Further, there is a need for a device and a method that provides a simple, efficient, and cost-effective diagnostic hearing assessment utilizing data compiled by a healthcare provider and analysis by a remotely-located ear specialist, without requiring a patient to visit each separately.

### BRIEF DESCRIPTION

The disclosure generally describes a system that enables a healthcare provider to administer a diagnostic hearing assessment, gather relevant clinical history, and capture findings on relevant physical examination and to consult with a remotely-located ear specialist. According to an aspect, the system includes an otoscope for capturing a diagnostic image of a target area of an ear canal, ear drum and/or ear tissue of a patient, and a telemedicine device that is communicably connected to the otoscope. The telemedicine device and the otoscope may generate a report that includes the diagnostic image and clinical history data of the patient. In an embodiment, the telemedicine device includes a data processor that generates the report, and transmits a consultation request to the remotely-located ear specialist. Since the consultation request includes the diagnostic image and the clinical history data of the patient, the remotely-located ear specialist can assess the patient's hearing as if they had actually had an opportunity to visualize the patient's render an opinion utilizing the information compiled by the healthcare provider.

The present disclosure further describes a method of administering a diagnostic hearing assessment and seeking a remote consultation with a remotely-located ear specialist. The method includes capturing a diagnostic image of a target area of a patient's ear using an otoscope. According to an aspect, the target area is selected by a healthcare provider, and includes at least one of the patient's ear canal, ear drum, and ear tissue. The method may further include generating a report that includes a preliminary hearing health assessment, the diagnostic image, as well as a clinical history data of the patient. Once the report has been generated, a request for the remote consultation to the ear specialist is submitted using a telemedicine device. According to an aspect, the method includes receiving an expert diagnosis report and a treatment plan from the remotely-located specialist. The expert diagnosis report and the treatment plan may be based on the preliminary hearing health assessment, the diagnostic image, and the clinical history data of the patient, as provided by the healthcare provider.

### BRIEF DESCRIPTION OF THE FIGURES

A more particular description of the system and method briefly described above will be rendered by reference to specific embodiments thereof that are illustrated in the appended drawings. Understanding that these drawings depict only typical embodiments and are not therefore to be considered to be limiting of its scope, exemplary embodiments will be described and explained with additional specificity and detail through the use of the accompanying drawings in which:

FIG. 1 schematically illustrates a system to administer a patient's hearing assessment and consult with a remotely-located ear specialist, according to an aspect; and

FIG. 2 is a flow chart illustrating a method of administering a diagnostic hearing health assessment and requesting a remote consultation with a remotely-located ear specialist, according to an embodiment.

Various features, aspects, and advantages of the embodiments will become more apparent from the following detailed description, along with the accompanying figures in which like numerals represent like components throughout the figures and text. The various described features are not necessarily drawn to scale, but are drawn to emphasize specific features relevant to embodiments.

### DETAILED DESCRIPTION

Reference will now be made in detail to various embodiments. Each example is provided by way of explanation, and is not meant as a limitation and does not constitute a definition of all possible embodiments.

In an embodiment, a system, including various devices and/or mechanisms, and a method that enables a healthcare provider to administer a diagnostic hearing assessment and consult with a remotely-located ear specialist is provided. As used herein, an “ear specialist” includes a medical professional, e.g., an osteopathic or medical physician specialized in diagnosis and treatment of the ear, including assessing and treating hearing loss. A “healthcare provider” is a provider of health related services that may or may not be also be a hearing specialist, e.g., a primary care physician, internal medicine physician, family medicine physician, geriatric physician, gynecologist, audiologist, hearing-aid kiosk operator, neurologist, naturopath doctor, chiropractic doctor and the like. By “expert diagnosis,” what is meant is the result of an assessment conducted by an ear specialist who has received specialized training in determining and treating health-related issues of the ear. By “interactive”, what is meant is of or pertaining to a two-way system of electronic communications by means of a computer. By “portable”, what is meant is capable of being easily carried or conveyed by hand. The configuration described herein is capable of providing a “hearing assessment”, an assessment of the person's sense of hearing that is not quite as rigorous as a traditional, full-blown hearing test performed by an audiologist or other ear specialist using an audiometer, but is sufficient to provide at least a baseline indication of hearing issues so that the patient can be referred to an ear specialist for further analysis. By “remotely-located”, what is meant is a location that is separate/different from the location/(s) of the healthcare provider and the patient, which can be a medical office that is separate from the healthcare provider's medical office.

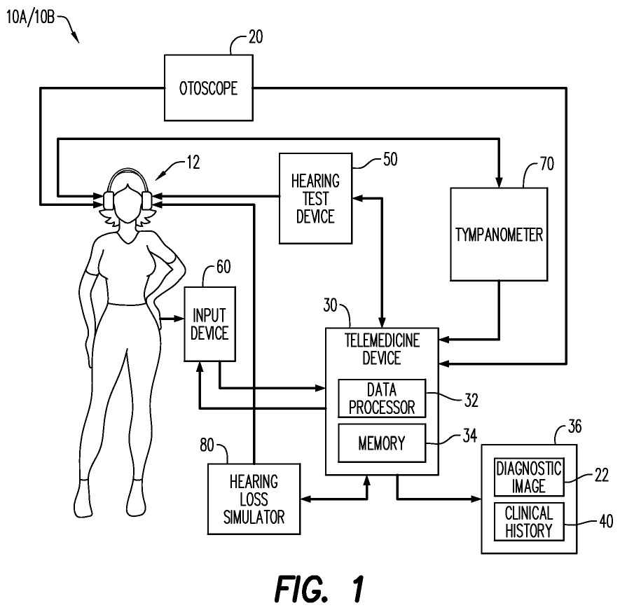

Now referring to FIG. 1 and according to an embodiment, a machine, kit or system 10A/10B is described. The systems 10A/10B enable a healthcare provider to administer a diagnostic hearing assessment to a patient or test subject 12, and consult with a remotely-located ear specialist. As shown herein, the system 10A/10B may include several components/devices, each of which may be operable to administer the diagnostic hearing assessment and/or facilitate the request for the remote consultation. Such components are illustrated in FIG. 1 as separate individual parts, although it would be understood by one of ordinary skill in the art that one or more of the devices/parts could be combined into a single device as described in more detail below.

FIG. 1 illustrated a system 10A that enables a healthcare provider to administer a diagnostic hearing assessment, includes a device and system to administer a portable interactive patient hearing assessment as generally described in U.S. application Ser. No. 14/190,924, filed Feb. 26, 2014, and U.S. Provisional Application No. 61/769,449, filed Feb. 26, 2013, each of which are incorporated herein by reference, in their entireties. The system 10A as described herein advantageously enables the healthcare provider to consult with a remotely-located ear specialist. In an embodiment, the system 10A electronically connects the healthcare provider with the remotely-located ear specialist, while providing the ear specialist with information that helps to diagnose a medical condition and render an opinion and treatment plan and to share it with the healthcare provider. Thus, the ear specialist may provide an expert diagnosis and treatment plan, and the system 10A is configured to receive the expert diagnosis and treatment plan from the remotely-located ear specialist. As an example, it is possible that the healthcare provider, e.g. an audiologist, is not co-located with the ear specialist, e.g. an otolaryngologist. Thus, the healthcare provider is provided with a tool that ultimately benefits the patient, by being able to provide the patient with an efficient (in both time and cost) diagnosis and plan for treating the issue, without the need for the patient to drive from one office to another, and/or to possibly even have to wait days to see the ear specialist, to get the treatment he/she needs.

The system 10A may be configured to provide information that includes results of hearing tests conducted and/or images (still or live) gathered by the healthcare provider, as well as health/clinical history data of the patient 12. Once the remotely-located ear specialist has completed a review of the information, the ear specialist may finalize his/her opinion, and provide the opinion and a recommended treatment plan to the healthcare provider.

According to aspect, the system 10A includes an otoscope 20, and a telemedicine device 30 that is communicably connected to the otoscope 20. The otoscope may capture a diagnostic image 22 of a target area of an ear canal, eardrum (tympanic membrane) and/or ear tissue of the patient 12. While various otoscopes 20 may be utilized, it is contemplated that the otoscope 20 of the system 10A includes a flexible extension that is able to extend into the patient's 12 ear in order to capture the diagnostic image 22. In an embodiment, the diagnostic image 22 is one of a static image and a video image. A video image may be particularly used when, for example, the otoscope 20 supplies a stream of air into the patient's ear canal that moves the patient's eardrum. The video image may help capture any potential damage to the eardrum, including one of more of perforations, liquid discharge, and the like. The captured diagnostic still or live images may help the remotely-located ear specialist provide a more accurate opinion, and provide the appropriate treatment plan for the patient 12. In an embodiment, the otoscope 20 is one of a wired device and a wireless device, each of which may be configured to capture a high definition (HD) color image (i.e., the diagnostic image 22). The HD color images of the patient's 12 ear canal, eardrum and/or ear tissue may be archived/stored for later viewing by the healthcare provider and/or the remotely-located ear specialist.

The otoscope 20 may be connected to the telemedicine device 30 in such a manner that allows the telemedicine device 30 to provide copies of the diagnostic images 22 to the remotely-located ear specialist. The otoscope 20 may be connected to the telemedicine device 30 via a USB, a wired, and/or a wireless (Wi-Fi) connection. According to an aspect, the telemedicine device 30 is one of a desktop computer, a personal computer, and a hand-held mobile digital electronic portable device.

In an embodiment, the telemedicine device 30 includes a data processor 32 and a memory 34. The data processor 32 may be configured to process the diagnostic image 22, and to generate a report 36 that includes the diagnostic image 22 and the clinical history data 40 of the patient 12, while the memory 34 stores the diagnostic image 22, along with the clinical history data 40. According to an aspect, the data processor 32 may also to transmit the consultation request, which includes both the diagnostic image 22 and the clinical history data 40, to the remotely-located ear specialist. Once the remotely-located ear specialist has reviewed and/or analyzed the information submitted along with the consultation request, the health specialist may send the diagnosis and the treatment plan to the healthcare provider. In an embodiment, the data processor 32 also receives the diagnosis and the treatment plan from the remotely-located ear specialist, and transmits them to the healthcare provider. The diagnosis may indicate whether the patient's 12 hearing is considered normal or abnormal, and when the patient's hearing is abnormal, the treatment plan is provided. Such treatment plans may include prescribing medication, ordering imaging studies such as computerized tomography of the temporal bone of the skull, referral for physical examination by the specialist, and the like.

In an embodiment, the clinical history data 40 includes the results of various previously-conducted tests to assess the hearing and/or ear health of the patient 12. Such data 40 may include at least one of a result of a pressure test, a tuning fork test, and a threshold hearing test. According to an aspect, the system 10A includes a tuning fork 90 that conducts the tuning fork test to test for conductive hearing loss in the patient 12, as well as a tympanometer 70 that conducts and indicates the results of the pressure test.

In an embodiment, the threshold hearing test is conducted by the healthcare provider. According to an aspect, the system 10A includes a hand-held, mobile hearing test device 50 for administering the threshold hearing test. The hand-held, mobile hearing test device 50 is configured to transmit at least four distinct pure tone frequencies, at varying sound levels, to the patient 12, in a way that repeatedly measures a threshold for each ear of the patient 12. According to an aspect, the hearing test device 50 is configured to transmit at least six distinct pure tone frequencies. The distinct pure tone frequencies may include 250 Hertz (Hz), 500 Hz, 1000 Hz, 2000 Hz, 4000 Hz and 8000 Hz. By “threshold” what is meant is the methodology of providing varying levels of loudness to identify a level of hearing perceived by the patient 12. For instance, as a starting point, a higher threshold sound is transmitted to the patient 12, and the patient 12 indicates that the sound has been received/perceived (as discussed in greater detail below). The threshold may thereafter be dropped to a level of loudness that would normally be considered too low for hearing, and the threshold is gradually raised until the patient 12 once again indicates that the sound has been heard. Then, the threshold is lowered once again, and gradually raised until the patient 12 once again indicates hearing. The threshold level is recorded accordingly to measure the level of hearing of the patient 12.

According to an aspect, the pure tone frequencies are sent to a headset that is worn by the patient 12 (not shown). The headset may include a wired connection to the hand-held, mobile hearing test device 50. In an embodiment, the headset is a Sennheiser HDA300 audiometer headphone.

In order to withstand rigor under US Food and Drug Administration procedures, and more importantly to qualify as an event that is reimbursable under personal health insurance plans, the hand-held, mobile hearing test device 50 must provide the hearing test in a repeatable, calibrated way. In other words, the hand-held, mobile hearing test device 50 is capable of being calibrated and tested for calibration from time to time as necessary so that the various frequency tones and loudness of those tones are uniform across all devices. Accordingly, the hand-held, mobile hearing test device 50 may interact with a calibration device as necessary for calibration (not shown).

According to an aspect, the system 10A includes a hand-held, mobile input device 60. The hand-held, mobile input device 60 may request that the patient 12 respond to one or more questions designed to solicit background information about the patient 12. Such background information includes, but is not limited to, identifying demographic data and clinical history pertinent to issues surrounding the ear, hearing and balance. The hand-held, mobile input device 60 may include, for example, a display (including e.g. a touch screen), a keyboard, a mouse or other pointing device, a switch/button, and componentry capable of providing output through the display, a speaker, and/or a printer, and is in communication with a data processor 32 (as described in more detail below).

According to an aspect, the hand-held, mobile input device 60 is configured for activation by the patient 12 upon the patient's hearing of the at least four pure tone frequencies transmitted by the hand-held, mobile hearing test device 50. The patient 12 is able to respond to the lowest sound level they can identify by activating the hand-held, mobile input device 60. In other words, the patient 12 is able to respond to each pure tone frequency (i.e., the sound/(s) actually heard by the patient 12), by providing input to the hand-held, input device 60. According to an aspect, the hand-held, mobile input device 60 also transmits a signal that is indicative of the patient's response, to the data processor 32.

In an embodiment, the hand-held, mobile input device 60 is a portable electronic device that is configured to host a plurality of modules/software. The modules may form at least a portion of a platform for conducting the hearing test, and may be downloadable onto the input/interface device 60, such as a personal or laptop computer, or onto any device, such as a hand-held mobile digital electronic portable device, a tablet, a telephone, or the like, whether or not such devices have interne access. In other words, the hand-held, mobile input device 60 and the mobile, hearing test device 50 may be integrated into a single unit. In an embodiment, the telemedicine device 30, the hand-held, mobile, hearing test device 50, and the handheld, mobile input device 60 are integrated into the single unit; therefore, the data processor 32 and memory 34 may be included in the single unit. As would be understood by one of ordinary skill in the art, activation of the hand-held, mobile input device 60 could be performed in many ways. In an embodiment, the hand-held, mobile input device 60 includes a switch/button that may be activated. In another embodiment, the patient 12 may touch and/or click on the screen of the device 60 (for instance, on the screen of a tablet or iPad®). In the embodiment where a button is used, the button may be connected to a device that is wired to the hand-held, mobile input device 60 (not shown), as would be understood by one of ordinary skill in the art.

In an embodiment, the platform is made available to users via cloud computing. As used herein, the term “cloud computing” refers to when tasks are assigned to a combination of connections, software, and services, accessed over a network. The network of servers and connections is collectively known as “the cloud.” Computing at the scale of the cloud allows users to access supercomputer-level power from a portable electronic device. Using an access point like an iPhone® or iPad® device, available from Apple, Inc., an Android® tablet available from Samsung, a Windows® operating system available from Microsoft or a personal computer (PC) using Windows® operating system from any PC manufacturer, users can reach into the cloud for resources as they need them. It would be understood by one of ordinary skill in the art that software can be provided that is compatible with Android (an operating system based on Linux), Apple® iOS and Windows® operating systems as well so that a thin client may be adapted for a variety of environments. Alternatively, various applications could simply be hosted or housed upon the device.

In any event, a signal is transmitted to the data processor 32, the signal being indicative of the patient's response to the specific pure tone frequency provided. The processor 32 may be configured to process and issue signals associated with the hearing test and to receive the response from the patient 12 based on the patient's ability to hear each of the at least four pure tone frequencies. As described above in relation to the hand-held, mobile hearing test device 50, the data processor 32 may similarly be housed on a portable device, such as an iPad®.

The system 10A may include a hearing loss simulator 80. The hearing loss simulator 80 may be configured to audibly demonstrate an effect of a hearing loss of the patient 12 as measured by the hand-held, mobile hearing test device 50 to a person accompanying the patient 12. The hearing loss simulator 80 may be included in the modules. In any event, once the results of the hearing test are processed by the data processor 32, the results are transmitted to a simulator 80, which is configured to visually display the hearing loss and audibly demonstrate the effects of the hearing loss measured by the hearing test. In short, the data processor 32 sends a signal indicative of whether or not the patient's hearing is considered normal or abnormal.

In an embodiment, the simulator 80 displays the following information to the patient and/or healthcare provider:

If a hearing loss is indicated that is of a high frequency hearing loss, the hearing loss simulator 80 will demonstrate to the patient what can be heard from, for instance, a woman as if their hearing is normal, as compared to what the patient is currently hearing. The demonstration via the simulator 80 can be repeated as necessary for the patient 12, the healthcare provider, and/or anyone accompanying the patient, like a spouse, parent or child, so that the person accompanying the patient 12 may get a sense for what the patient 12 is not hearing. In other words, the person accompanying the patient 12 may get a scope of the patient's 12 hearing loss.

If a hearing loss is indicated that is of a low frequency, a similar demonstration can be made to that of the high frequency hearing loss discussed above, using a male voice that is deeper in timber and has a low frequency of sound. For all other types of hearing loss, a demonstration may be made to the patient 12 of what can be heard from a woman if their hearing is normal, as compared to what the patient 12 him/herself is hearing. Of course, any of these results are capable of being repeated for the patient 12, the person accompanying the patient 12, and/or anyone else having an interest in the results of the hearing test.

If the hearing test is determined to be abnormal, then an additional test will be conducted in the form of the pressure test. In an embodiment, the tympanometer 70 is provided to conduct a middle ear compliance test in at least one ear of the patient 12. Tympanometry is an examination used to test the condition of the middle ear and mobility of the eardrum (tympanic membrane) and the conduction bones by creating an air pressure variation of the ear canal. The pressure test may serve as an objective means of testing the middle-ear functions, and should be viewed in conjunction with the pure tone audiometry for an overall test of hearing. In evaluating hearing loss, tympanometry permits a distinction between the presence or absence of middle ear interference with respect to a measured hearing loss. Furthermore, in a primary care setting, a tympanometer 70 is typically useful for diagnosing otitis media by demonstrating the presence of a middle ear effusion. In any event, the tympanometer 70 is configured to transmit a signal indicative of the results of the pressure test to the processor 32.

Upon receipt of the patient's response to the hearing test described above and also of the pressure test results, the processor 32 provides the results of the assessment to the healthcare provider so that the patient 12 may be referred to an appropriate remotely-located ear specialist as required. In an embodiment, the results include a report and recommendation regarding the patient's hearing, which can be provided in a display and/or a printed report. If no hearing loss is detected, the system may simply provide the results as a printed report for the physician and/or the patient 12. If a symmetrical hearing loss is detected from the hearing test, and the results of the pressure test is normal bilaterally, and a physical exam of the ear is normal bilaterally, the processor 32 may recommend a referral to an audiologist. For all other combinations of results, a referral to an otolaryngologist is generated.

In yet a further embodiment, a listing of qualified remotely-located ear specialists is stored in a database, (not specifically shown), housed either on the data processor 32 itself, the memory 34 of the telemedicine device 30, or is accessible by the internet to a cloud-based database, so that proper referrals can be made. Such a database could be maintained by the healthcare provider. Alternatively, the database is maintained based on qualifying criteria, so that referrals may be made with confidence.

Further embodiments of the disclosure relate to a system 10B that enables a healthcare provider to administer a portable interactive patient hearing assessment and to consult with a remotely-located otolaryngologist. Since otolaryngologists have specialized training in assessing and treating disorders and diseases related to the ear, nose and throat, and are required to maintain yearly certifications, otolaryngologists are oftentimes more equipped than healthcare providers to treat ear disorders and/or hearing loss. According to an aspect, the system 10B includes several components, including an otoscope 20, a telemedicine device 30, a hand-held, mobile hearing test device 50, a hand-held, mobile input device 60, a tuning fork 90, a tympanometer 70, and a hearing loss simulator 80. Each of the components are described above in connection with the system 10A, thus, for purposes of convenience and not limitation, the various features, attributes, and properties, and functionality of each of the components therein discussed in connection with system 10A are not repeated here. Differences between the system 10A that enables the healthcare provider to administer a diagnostic hearing assessment and to consult with a remotely-located ear specialist, and the system 10B that enables the healthcare provider to administer the portable interactive patient hearing assessment and to consult with a remotely-located otolaryngologist will be elaborated below.

In an embodiment, the system 10B provides a report and/or a recommendation to indicate a hearing health summary of the patient 12. When the report includes a hearing health summary that indicates a normal hearing capacity of the patient, the report may be provided to the healthcare provider and/or the patient 12. According to an aspect, when the hearing health summary indicates an abnormal hearing capacity of the patient 12, the report and a request for a remote consultation is provided to the remotely-located otolaryngologist. The transmission of the report and/or recommendation may be coordinated/managed by the telemedicine device 30.

In an embodiment, the telemedicine device 30 is communicably connected to each of the otoscope 20 and the mobile input device 60. In this arrangement, the telemedicine device 30 includes a data processor 32, which is configured to process respective signals transmitted by the otoscope 20 and the hand-held, mobile input device 60. The respective signal transmitted by the otoscope may include a diagnostic image 22 of the target area of the patient ear, while the respective signal transmitted by the hand-held, mobile input device 60 may be indicative of the patient's response regarding his/her ability to hear each of the at least four pure tone frequencies transmitted by the hand-held, mobile hearing test device 50.

The data processor 32 may also prepare the report and the recommendation to indicate the hearing health summary of the patient 12, and in some instances, includes the request for the remote consultation. According to an aspect, the abnormal hearing capacity included in the report to the otolaryngologist is indicative of at least one of a symmetrical hearing loss, and an abnormal pressure test result. Upon receipt of the request for consultation, the remotely-located otolaryngologist reviews the patient's hearing health summary and the diagnostic image 22 captured by the otoscope 20, and may then send the results of his/her analysis to the healthcare provider. The processor 32 may be configured to receive the results, including a diagnosis report and/or a treatment plan, from the remotely-located otolaryngologist in response to the request for the remote consultation.

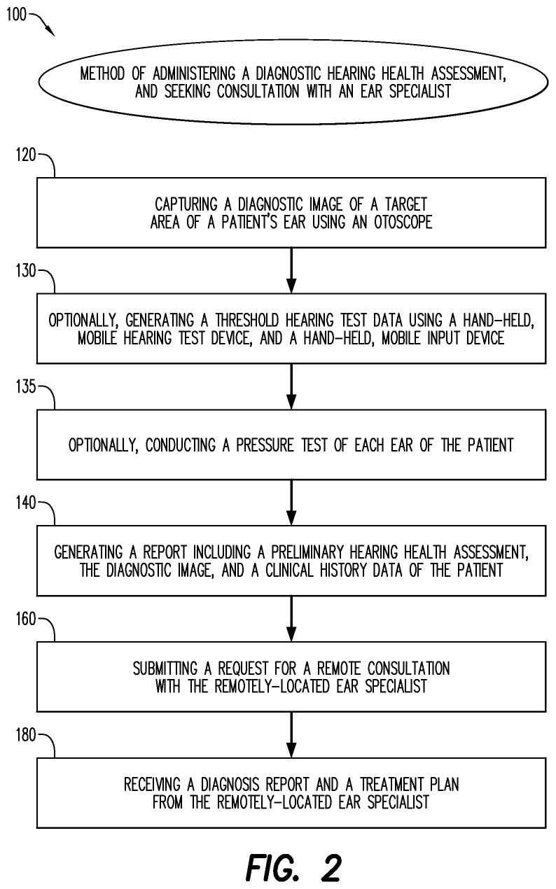

Now referring to FIG. 2, a method 100 of administering a diagnostic hearing assessment and seeking a consultation with a remotely-located ear specialist is illustrated. The method 100 includes a system 10A/10B that enables the healthcare provider to submit an automatic request for consultation/remote consultation that includes the results of a preliminary heating health assessment, and diagnostic images of parts of a patient's inner ear, with the remotely-located ear specialist. While each component described hereinabove with reference to FIG. 1 may be included in the method 100 steps, for convenience, they are not repeated.

According to an aspect, the method 100 includes capturing 120 the diagnostic image of a target area of a patient's ear using an otoscope. The otoscope utilized may be substantially as described hereinabove, with reference to FIG. 1. In an embodiment, the target area selected by the healthcare provider includes a portion of the inner ear of the patient 12. The target area capture may include at least one of the patient's ear canal, eardrum, and ear tissue. The diagnostic image may include a still image and/or a live image. In some instance, live images of the inner ear may be particularly helpful to provide the remotely-located ear specialist with adequate views of the ear tissue to check for abnormal growths and/or tumors. Live images may also provide adequate view of the tympanic membrane to assess whether the tympanic membrane has ruptured, which may lead to hearing loss.

The diagnostic hearing assessment may include the results of additional tests conducted by the healthcare provider, such as, for example, a threshold hearing test and a pressure test. According to an aspect, the method includes generating 130 a threshold hearing test data using a hand-held, mobile hearing test device and a hand-held, mobile input device. The hand-held, mobile hearing test device may transmit at least four distinct pure tone frequencies to the patient, and the hand-held, mobile input device may be activated by the patient in response to the patient hearing the at least four pure tone frequencies. The method may further include conducting 135 a pressure test of each ear of the patient, using a tympanometer. The method may include generating 140 a report including a preliminary hearing health assessment, the diagnostic image, and a clinical history data of the patient. The preliminary hearing health summary may include the results of the pressure test and the threshold hearing test.

According to an aspect, the hearing healthy summary is indicative of one of a normal hearing capacity and an abnormal hearing capacity. If an abnormal hearing capacity and/or a questionable normal hearing capacity is included in the hearing health summary, the method 100 may include submitting 160 a request for the remote consultation to the remotely-located ear specialist. The request may be sent using a telemedicine device, and may include the preliminary hearing health assessment and the diagnostic image.

According to an aspect, the preliminary hearing health assessment includes details of the patient's clinical medical and/or hearing health history, such details including at least one a result of the threshold hearing test, a pure tone audiometry test, a tympanometry test, and a tuning fork test. The telemedicine device utilized may be communicably connected to the otoscope, such that the healthcare provider may access the diagnostic images captured, and submit of copies of each to the ear specialist, using just the telemedicine device. The telemedicine device may include a data processor and a memory. The data processor may process a signal transmitted by the otoscope, prepare the preliminary assessment summary, and submit the request for the remote consultation. While the request may be sent soon after the patient's information (e.g., demographic data, clinical history, preliminary hearing health assessment) is compiled, the patient's information may be stored for some time in the memory.

In an embodiment, the method further includes receiving 180, by the healthcare provider, a diagnosis report and a treatment plan from the remotely-located ear specialist. The method 100 and systems 10A/10B described herein helps to broaden the reach of remotely-located ear specialists, such as otolaryngologists, and help connect them with healthcare providers so that they can assist patients in various geographical locations. Through the utilization of the method 100 and systems 10A/10B described hereinabove, the ear specialists, specifically trained to manage and treat patients with diseases and disorders relating to the ear, may provide more accurate diagnosis and effective treatment plans to patients, without requiring the patients to visit the physical location of the remotely-located ear specialist.

The systems and method illustrated are not limited to the specific embodiments described herein, but rather, features illustrated or described as part of one embodiment can be used on or in conjunction with other embodiments to yield yet a further embodiment. It is intended that the system and method include such modifications and variations. Further, steps described in the method may be utilized independently and separately from other steps described herein.

While the system and method have been described with reference to specific embodiments, it will be understood by those skilled in the art that various changes may be made and equivalents may be substituted for elements thereof without departing from the scope contemplated. In addition, many modifications may be made to adapt a particular situation or material to the teachings found herein without departing from the essential scope thereof

Embodiments can be implemented in digital electronic circuitry, or in computer hardware, firmware, software, or in combinations thereof. Components and/or devices can be implemented in a computer program product tangibly embodied or stored in a machine-readable storage device or non-transitory computer-readable medium, and method steps can be performed by the programmable data processor executing a program of instructions to perform functions by operating on input data and generating output. The method can be implemented in one or more computer programs that are executable on a programmable system including the at least one programmable data processor coupled to receive data and instructions from, and to transmit data and instructions to, a data storage system, at least one input device, and at least one output device. Each computer program can be implemented in a high-level procedural or object oriented programming language, or in assembly or machine language if desired; and in any case, the language can be a compiled or interpreted language.

Suitable data processors include, by way of example, both general and special purpose microprocessors. Generally, a processor will receive instructions and data from a read-only memory and/or a random access memory. Generally, a computer will include one or more mass storage devices for storing data files; such devices include magnetic disks, such as internal hard disks and removable disks; magneto-optical disks; and optical disks. Storage devices suitable for tangibly embodying computer program instructions and data include all forms of non-volatile memory, including by way of example semiconductor memory devices, such as EPROM, EEPROM, and flash memory devices; magnetic disks such as internal hard disks and removable disks; magneto-optical disks; and, CD_ROM disks. Any of the foregoing can be supplemented by, or incorporated in, ASICs (application-specific integrated circuits).

In this specification and the claims that follow, reference will be made to a number of terms that have the following meanings. The singular forms “a,” “an” and “the” include plural referents unless the context clearly dictates otherwise. Furthermore, references to “one embodiment”, “some embodiments”, “an embodiment” and the like are not intended to be interpreted as excluding the existence of additional embodiments that also incorporate the recited features. Approximating language, as used herein throughout the specification and claims, may be applied to modify any quantitative representation that could permissibly vary without resulting in a change in the basic function to which it is related.

As used herein, the terms “may” and “may be” indicate a possibility of an occurrence within a set of circumstances; a possession of a specified property, characteristic or function; and/or qualify another verb by expressing one or more of an ability, capability, or possibility associated with the qualified verb. Accordingly, usage of “may” and “may be” indicates that a modified term is apparently appropriate, capable, or suitable for an indicated capacity, function, or usage, while taking into account that in some circumstances the modified term may sometimes not be appropriate, capable, or suitable. For example, in some circumstances an event or capacity can be expected, while in other circumstances the event or capacity cannot occur—this distinction is captured by the terms “may” and “may be.”

As used in the claims, the word “comprises” and its grammatical variants logically also subtend and include phrases of varying and differing extent such as for example, but not limited thereto, “consisting essentially of” and “consisting of.”

Advances in science and technology may make equivalents and substitutions possible that are not now contemplated by reason of the imprecision of language; these variations should be covered by the appended claims. This written description uses examples to disclose embodiments, including the best mode, and also to enable any person of ordinary skill in the art to practice the embodiments, including making and using any devices or systems and performing any incorporated methods. The patentable scope of the embodiments is defined by the claims, and may include other examples that occur to those of ordinary skill in the art. Such other examples are intended to be within the scope of the claims if they have structural elements that do not differ from the literal language of the claims, or if they include equivalent structural elements with insubstantial differences from the literal languages of the claims.

What is claimed is:

1. A system to enable a healthcare provider to administer a diagnostic hearing assessment and to consult with a remotely-located ear specialist, the system comprising:
an otoscope for capturing a diagnostic image of a target area of an ear canal, ear drum and/or ear tissue of a patient; and
a telemedicine device communicably connected to the otoscope, the telemedicine device comprising,
a data processor configured to process the diagnostic image, and
a memory configured to store the diagnostic image and a clinical history data of the patient, the clinical history data comprising a result of at least one of a threshold hearing test and a result of a pressure test of each ear of the patient, wherein
the data processor is configured to generate a report comprising the diagnostic image and the clinical history data, and to transmit a consultation request to the remotely-located ear specialist.

2. The system of claim 1, wherein the data processor is further configured to receive an expert diagnosis and a treatment plan from the remotely-located ear specialist.

3. The system of claim 1, further comprising:
a hand-held, mobile hearing test device configured for administration of the threshold hearing test by the healthcare provider, the hand-held, mobile hearing test device being configured to transmit at least four distinct pure tone frequencies to the patient; and
a hand-held, mobile input device configured for activation by the patient upon hearing the at least four pure tone frequencies transmitted by the hand-held, mobile hearing test device, and for transmission of a signal indicative of the patient's response to the data processor of the telemedicine device.

4. The system of claim 1, further comprising:
a tympanometer configured to conduct the pressure test and to indicate the results of the pressure test.

5. The system of claim 1, wherein the clinical history data further comprises a result of a tuning fork test conducted to test for conductive hearing loss in the patient.

6. The system of claim 2, wherein the expert diagnosis indicates whether the patient's hearing is considered normal or abnormal, and comprises the treatment plan when the patient's hearing is abnormal.

7. The system of claim 1, wherein the telemedicine device is one of a desktop computer, a personal computer, and a hand-held mobile digital electronic portable device.

8. The system of claim 1, wherein the diagnostic image is one of a static image and a video image.

9. The system of claim 3, further comprising:
a hearing loss simulator configured to audibly demonstrate an effect of a hearing loss of the patient as measured by the hand-held, mobile hearing test device to a person accompanying the patient.

10. A system to enable a healthcare provider to administer a portable interactive patient hearing assessment and to consult with a remotely-located otolaryngologist, the system comprising:
a hand-held, mobile hearing test device for administration of a threshold hearing test by the healthcare provider, wherein the hand-held, mobile hearing test device is configured to transmit at least four distinct pure tone frequencies to the patient;
a hand-held, mobile input device configured for activation by the patient upon hearing the at least four pure tone frequencies, wherein the activation indicates a result of the threshold hearing test;
an otoscope configured to capture and store a diagnostic image of a target area of each ear of the patient;
a tympanometer configured to conduct a pressure test of each ear of the patient and to indicate a result of the pressure test; and
a telemedicine device communicably connected to each of the otoscope and the mobile input device, the telemedicine device comprising a data processor, wherein the data processor is configured to process respective signals transmitted by the otoscope and the hand-held, mobile input device, and to prepare a report and a recommendation to indicate a hearing health summary of the patient, the hearing healthy summary being indicative of one of a normal hearing capacity and an abnormal hearing capacity, wherein
when the hearing health summary of the patient comprises the normal hearing capacity, the system provides the results in the report to the healthcare provider and/or to the patient, and
when the hearing health summary of the patient comprises the abnormal hearing capacity, the system provides the results in the report to the remotely-located otolaryngologist, and the recommendation comprises a request for a consultation with the remotely-located otolaryngologist.

11. The system of claim 10, wherein the processor is configured to receive a diagnosis report and a treatment plan from the remotely-located otolaryngologist, in response to the request for the consultation.

12. The system of claim 10, wherein the respective signal transmitted by the otoscope comprises the diagnostic image of the target area, and wherein the respective signal transmitted by the hand-held, mobile input device is indicative of the patient's response regarding the patient's ability to hear each of the at least four pure tone frequencies.

13. The system of claim 12, wherein the abnormal hearing capacity is indicative of at least one of a symmetrical hearing loss, and an abnormal pressure test result.

14. The system of claim 10, wherein the telemedicine device further comprises a memory to store the diagnostic image, the result of the threshold hearing test, and the result of the pressure test.

15. The system of claim 10, further comprising:
a hearing loss simulator configured to audibly demonstrate an effect of a hearing loss of the patient as measured by the hand-held, mobile hearing test device to a person accompanying the patient.

16. A method of administering a diagnostic hearing assessment and seeking a remote consultation with a remotely-located ear specialist, the method comprising:
capturing a diagnostic image of a target area of a patient's ear using an otoscope, the target area being selected by a healthcare provider and comprising at least one of the patient's ear canal, ear drum, and ear tissue;
generating a report comprising a preliminary hearing health assessment, the diagnostic image, and a clinical history data of the patient;
submitting a request for the remote consultation to the remotely-located ear specialist using a telemedicine device, the request comprising the preliminary hearing health assessment; and
receiving an expert diagnosis report and a treatment plan from the remotely-located specialist.

17. The method of claim 16, wherein the preliminary assessment summary comprises a clinical history of the patient, the clinical history comprising at least one a result of a threshold hearing test, a pure tone audiometry test, a tympanometry test, and a tuning fork test.

18. The method of claim 16, further comprising:
generating a threshold hearing test data using a hand-held, mobile hearing test device and a hand-held, mobile input device, wherein the hand-held, mobile hearing test device is configured to transmit at least four distinct pure tone frequencies to the patient when activated by the healthcare provider, and the hand-held, mobile input device is activated by the patient upon hearing the at least four pure tone frequencies.

19. The method of claim 16, wherein the telemedicine device is communicably connected to the otoscope, and wherein the telemedicine device comprises a data processor configured to process the diagnostic image, and a memory configured to store the diagnostic image and the clinical history data of the patient.

20. The method of claim 19, wherein the data processor is configured to process a signal transmitted by the otoscope, prepare the preliminary assessment summary and submit the request for the remote consultation.
